(19) United States
(12) Patent Application Publication
(10) Pub. No.: US 2013/0115172 A1
(43) Pub. Date: May 9, 2013

(54) MAGNETIC NANOPHOSPHOR HAVING CORE/SHELL STRUCTURE AND THE SYNTHETIC METHOD THEREOF

(71) Applicant: Korea Institute of Science and Technology, Seoul (KR)

(72) Inventors: Ho Seong JANG, Seoul (KR); Kyoungja WOO, Seoul (KR); Kipil LIM, Seoul (KR)

(73) Assignee: KOREA INSTITUTE OF SCIENCE AND TECHNOLOGY, Seoul (KR)

(21) Appl. No.: 13/670,870

(22) Filed: Nov. 7, 2012

(30) Foreign Application Priority Data
Nov. 8, 2011 (KR) 10-2011-0115669

(51) Int. Cl. C09K 11/77; A61K 49/08

(52) U.S. Cl. CPC C09K 11/7772; A61K 49/08; Y10S 977/773; Y10S 977/892; B82Y 40/00
USPC 424/9.32; 252/62.55; 427/127; 977/773; 977/892; 977/930

(57) ABSTRACT

The present invention relates to a nanophosphor and method for synthesizing the same, and provides a nanophosphor containing fluoride-based nanoparticles co-doped with Yb³⁺ and Er³⁺ expressed by the following Chemical Formula 1,

NaY_{1-w-z-x-y}Gd_{w}L_{z}F₄:Yb³⁺ₓ,Er³⁺_{y} (1)

wherein, the description of the values x, y, w, z, and L is the same as defined above.

The nanophosphor may exhibit an excellent luminous intensity despite having a small particle size, and be excited by infrared rays to emit visible light, and have magnetic properties and thus can be used as a contrast agent, a counterfeit prevention code, and the like.

### RELATED APPLICATION

This application claims the benefit of priority to Korean Patent Application No. 10-2011-0115669, filed on Nov. 8, 2011, which is hereby incorporated by reference for all purposes as if fully set forth herein.

### BACKGROUND OF THE INVENTION

1. Field of the Invention

The present invention relates to a nanophosphor and method for synthesizing the same, and more particularly, to a fluoride-based nanophosphor excited by infrared rays to emit visible light, having magnetic properties, and being useful for used for a magnetic resonance imaging (MRI) and fluorescent contrast agent and method for preparing the same.

2. Background of the Invention

Since the Bawendi group at MIT had reported a method of synthesizing CdSe nanoparticles having a uniform size distribution (C. B. Murray et al. J. Am. Chem. Soc., vol. 115, pp. 8706-8715 (1993)), studies on nanoparticles have been actively carried out.

Unlike semiconductor nanocrystals, photoluminescent nanoparticles doped with a lanthanide element have a property in which peak position of a luminescence spectrum does not change even when the size of particles changes.

It is because the emission of photoluminescent nanoparticles, namely, nanophosphor doped with a lanthanide element, is generated by 4f electron transition of the lanthanide element such as 4f-4f or 4f-5d transitions. Accordingly, when photoluminescent nanoparticles doped with a lanthanide element are used, it has the advantage of maintaining a desired emission wavelength even if the size of particles varies when the need arises.

The emission of a nanophosphor typically occurs when excited by ultraviolet rays or visible light. However, if the light of short wavelength such as ultraviolet rays or visible light is used as an excitation source when the nanophosphor is used as a contrast agent for biological imaging, cells or living tissues are susceptible to damage.

Light generated by the emission of the nanophosphor can penetrate to a shallow depth in biological tissues, and thus the location of the contrast agent capable of receiving excited energy to emit light will be constrained.

In addition, due to fluorescence exhibited from cells or biological tissues themselves, it is difficult in in vivo imaging to distinguish a luminous signal generated from nanoparticles used as a contrast agent.

In order to overcome this, in recent years, studies for using infrared rays as excitation light have been carried out.

Bawendi et al. have shown that InAs/ZnCdS or the like capable of infrared emission can be used for imaging even at a deeper location within the cells [J. Am. Chem. Soc. vol. 132, 470-471 (2010)], and attempts for using up-conversion emission through the two-photon and three-photon absorption effect of metal nanoparticles [Angew. Chem. Int. Ed. vol. 49, 3485-3488 (2010)] or quantum dots [Nano Lett. vol. 10, 5109 to 5115 (2010)] have been also carried out.

However, the above studies have disadvantages in that they are involved with the use of harmful heavy metals such as As or Cd and expensive laser light sources should be used due to a very low efficiency of up-conversion emission through the two-photon and three-photon absorption excitation.

On the contrary, NaYF₄ particles co-doped with ytterbium (Yb) and erbium (Er) have a property in that they are excited by near-infrared rays to emit visible light and thus may be a very suitable material as a contrast agent for biological imaging.

In particular, NaYF₄ is known as the most efficient matrix exhibiting up-conversion [Chem. Mater. vol. 16, 1244 to 1251 (2004)]. Nanophosphor NaYF₄:Yb, Er exhibits an up-conversion phenomenon through allowing ytterbium to absorb externally applied near infrared rays to transfer the energy to erbium, thereby exhibiting very high efficiency compared to the up-conversion phenomenon due to two-photon absorption.

Due to the foregoing differences in light-emitting mechanism, when nanophosphor NaYF₄:Yb, Er is used as a contrast agent, it may be possible to implement up-conversion emission with a low-cost diode laser without using a high cost pulse laser apparatus.

However, NaYF₄ exhibits polymorphism between an α-phase cubic structure and a β-phase hexagonal structure, of which only β-phase shows a property exhibiting excellent performance of the up-conversion emission.

At this time, it requires a very high synthesis temperature to obtain β-phase NaYF₄, and thus not suitable for biomedical applications because the size of synthesized particles increases.

Specifically, previously reported up-conversion nanophosphor NaYF₄:Yb, Er exhibits the size of at least 20 nm, and thus has a limitation in using as a contrast agent for biological imaging [J. Phys. Chem. C vol. 111, from 13,730 to 13,739 (2007)].

Accordingly, the development of nanoparticles having a small size while exhibiting strong up-conversion emission is strongly needed.

### SUMMARY OF THE INVENTION

An object of the present invention is to provide a nanophosphor capable of being excited by infrared rays to emit visible light so as to be applicable as a fluorescent contrast agent, having magnetic properties to be applicable as a magnetic resonance imaging (MRI) contrast agent, and exhibiting an excellent luminous intensity even though having a small particle size. In addition, another object of the present invention is to provide a method of preparing a nanophosphor having a small particle size without changing the synthesis temperature.

A nanophosphor according to an embodiment of the present invention may comprise fluoride-based nanoparticles co-doped with Yb³⁺ and Er³⁺ expressed by the following Chemical Formula (I)

NaY_{1-w-z-x-y}Gd_{w}L_{z}F₄:Yb³⁺ₓ,Er³⁺_{y} (1)

In the Chemical Formula 1, x is a real number in the range of 0.1≦x≦0.9; y is a real number in the range of 0<y≦0.1; 0.1<x+y≦1; w is a real number in the range of 0≦w≦1′; and z is a real number in the range of 0≦z≦1; 0≦w+z≦1; and L is any one selected from the group consisting of lanthanide elements, and combinations thereof.

The nanophosphor may comprise a core containing the nanoparticles and a shell located on a surface of the core, and the shell may be composed of the following Chemical Formula 2,

NaGd₁₋ᵥMᵥF₄ (2)

In the Chemical Formula 2, v is a real number in the range of 0≦v<1; and M is any one selected from the group consisting of Y, lanthanide elements, and combinations thereof.

The lanthanide element may be any one selected from the group consisting of La, Ce, Pr, Nd, Pm, Sm, Eu, Gd, Tb, Dy, Ho, Tm and Lu.

In the Chemical Formula 1, x is a real number in the range of 0.1≦x≦0.4; y is a real number in the range of 0.001≦y≦0.05; and 0.101≦x+y≦0.45.

The size of nanoparticles may be 1 nm to 10 nm.

The nanoparticles may have a hexagonal structure.

The size of nanophosphor may be greater than 1 nm and equal to or less than 20 nm.

The nanophosphor may have up-conversion properties and magnetic properties.

A method of preparing a nanophosphor according to another embodiment of the present invention may comprise the steps of a mixture solution preparation step for preparing a first mixture solution containing a yttrium precursor, a ytterbium precursor, an erbium precursor, oleic acid and 1-octadecene; a complex formation step for heating the first mixture solution to form a solution containing a lanthanide complex; a reaction solution preparation step for mixing a solution containing the lanthanide complex with a second mixture solution containing a sodium precursor, a fluorine precursor and alcohol to prepare a reaction solution; and a nanoparticles formation step for removing alcohol from the reaction solution and performing a thermal treatment on the reaction solution from which alcohol is removed to form nanoparticles, wherein the nanoparticles are fluoride-based nanoparticles of the above Chemical Formula 1, co-doped with Yb³⁺ and Er³⁺.

The first mixture solution may further comprise a gadolinium precursor.

The yttrium precursor may be any one selected from the group consisting of yttrium acetate (Y(CH₃COO)₃), yttrium chloride (YCl₃), yttrium chloride hexahydrate (YCl₃.6H₂O), and combinations.

The ytterbium precursor may be any one selected from the group consisting of ytterbium acetate (Yb(CH₃COO)₃), ytterbium chloride (YbCl₃), ytterbium chloride hexahydrate (YbCl₃.6H₂O), and combinations thereof.

The erbium precursor may be any one selected from the group consisting of erbium acetate (Er(CH₃COO)₃), erbium chloride (ErCl₃), erbium chloride hexahydrate (ErCl₃.6H₂O), and combinations thereof.

The gadolinium precursor may be any one selected from the group consisting of gadolinium acetate (Gd(CH₃COO)₃), gadolinium chloride (GdCl₃), gadolinium chloride hexahydrate (GdCl₃.6H₂O), and combinations thereof.

The thermal treatment performed during the nanoparticles formation step may be carried out at temperatures of 200 to 370° C. for 30 minutes to four hours.

The nanophosphor preparation method may further comprise a cooling step for cooling the thermally treated nanoparticles, and a cleaning step for cleaning the cooled nanoparticles with acetone or ethanol, subsequent to the nanoparticles formation step.

The nanophosphor preparation method may further comprise a shell formation step subsequent to the nanoparticles formation step.

The shell formation step may comprise a shell solution preparation step for preparing a third mixture solution comprising sodium oleate and a lanthanide precursor comprising gadolinium precursor; a nanoparticles mixing step for performing a thermal treatment on the third mixture solution to form gadolinium oleate, dissolving the gadolinium oleate in a solution containing oleic acid, 1-octadecene, to which the nanoparticles that have been formed during the nanoparticles formation step are mixed to prepare a fourth mixture solution; a shell reaction solution preparation step for mixing a solution containing a sodium precursor, a fluorine precursor and alcohol with the fourth mixture solution to prepare a shell reaction solution; and a shell formation step for removing alcohol from the shell reaction solution followed by a thermal treatment to form a shell on the surface of core comprising the nanoparticles.

The gadolinium precursor may be any one selected from the group consisting of gadolinium acetate (Gd(CH₃COO)₃), gadolinium chloride (GdCl₃), gadolinium chloride hexahydrate (GdCl₃.6H₂O), and combinations thereof.

A contrast agent according to still another embodiment of the present invention may be a fluorescent or magnetic resonance imaging contrast agent containing the nanophosphor.

An infrared sensor according to yet still another embodiment of the present invention may comprise the nanophosphor.

A counterfeit prevention code according to still yet another embodiment of the present invention may comprise the nanophosphor.

A solar cell according to yet still another embodiment of the present invention may comprise the nanophosphor.

Unless otherwise specified in the present invention, the lanthanoids represent elements classified as a lanthanide series on the periodic table of elements, and more specifically represent any one selected from the group consisting of lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), and lutetium (Lu).

Hereinafter, the present invention will be described in more detail.

A nanophosphor according to an embodiment of the present invention may comprise fluoride-based nanoparticles co-doped with Yb³⁺ and Er³⁺ expressed by the following Chemical Formula 1.

NaY_{1-w-z-x-y}Gd_{w}L_{z}F₄:Yb³⁺ₓ,Er³⁺_{y} (1)

Wherein,

x is a real number in the range of 0.1≦x≦0.9; y is a real number in the range of 0<y≦0.1; 0.1<x+y≦1; w is a real number in the range of 0≦w≦1′; and z is a real number in the range of 0≦z≦1; 0≦w+z≦1; and L is any one selected from the group consisting of lanthanide elements, and combinations thereof.

In the Chemical Formula 1, the lanthanide element may be any one selected from the group consisting of La, Ce, Pr, Nd, Pm, Sm, Eu, Gd, Tb, Dy, Ho, Tm and Lu.

In the Chemical Formula 1, w may be a real number in the range of 0<w≦1.

In the Chemical Formula 1, z may be a real number in the range of 0<z≦1.

In the Chemical Formula 1, w and z may satisfy the condition of 0<w+z≦1

In the Chemical Formula 1, x may be a real number in the range of 0.1≦x≦0.4.

In the Chemical Formula 1, y may be a real number in the range of 0.001≦y≦0.05.

In the Chemical Formula 1, x and y may satisfy the condition of 0.101≦x+y≦0.45.

In case where x and y are within the above-mentioned ranges, a ratio of green emission peak to red emission peak can be enhanced, thereby obtaining an excellent green emission characteristic.

The size of nanoparticles may be less than 10 nm, or may be 1 to 10 nm. A nanophosphor containing the nanoparticles may have a size of less than 10 nm, and thus may be formed to have a much smaller size than that of the existing nanophosphor which has a size of larger than or equal to 20 nm, thereby being suitably applicable to in vivo imaging.

The nanoparticles may have a hexagonal structure. In case where fluoride-based nanoparticles co-doped with Yb³⁺ and Er³⁺ expressed by the above Chemical Formula 1 have a hexagonal structure, the nanophosphor having a high luminous strength can be obtained.

The nanophosphor may comprise a core containing the nanoparticles and a shell located on a surface of the core, and the shell may be composed of the following Chemical Formula 2,

NaGd₁₋ᵥMᵥF₄ (2)

Wherein,

v is a real number in the range of 0≦v<1, and may be a rear number in the range of 0≦v≦0.5;

M may be any one selected from the group consisting of Y, lanthanide elements, and combinations thereof, and said M may be any one selected from the group consisting of La, Ce, Pr, Nd, Pm, Sm, Eu, Gd, Tb, Dy, Ho, Tm and Lu.

The shell may have a crystalline structure, and may be formed by epitaxial growth.

In case where the shell is a crystalline shell which has been epitaxially grown, the surface defect of nanoparticles can be reduced, thereby obtaining excellent luminous properties.

The size of nanophosphor may be equal to or less than 20 nm, and may be greater than 1 nm and equal to or less than 20 nm. The nanophosphor may be implemented with a significantly small size even though having the structure of a core and a shell, thereby being suitably applicable to in vivo imaging.

The nanophosphor may have up-conversion properties, and may have magnetic properties.

The nanophosphor may comprise fluoride-based nanoparticles co-doped with Yb³⁺ and Er³⁺ having a high luminous intensity as its core to exhibit an excellent luminous characteristic even though having a small size of particles, and thus may be applicable to in vivo situations as well as in vitro situations.

Furthermore, the nanophosphor may comprise a core comprising the nanoparticles of Chemical Formula 1 and a shell expressed by the Chemical Formula 2, and thus up-conversion emission can be further increased even though having a small size of particles, thereby further increasing a luminous intensity of the nano luminous body itself.

The nanophosphor has magnetic properties along with the excellent luminescent properties, and thus can be applicable to a magnetic resonance imaging contrast agent as well as fluorescent contrast agent. Furthermore, the nanophosphor may be contained in an infrared sensor to allow a sensitive operation of the sensor, and applicable to a counterfeit prevention processing, and contained in a solar cell to enhance the efficiency of the solar cell.

A method of preparing a nanophosphor according to another embodiment of the present invention may comprise a mixture solution preparation step, a complex formation step, a reaction solution preparation step, and a nanoparticles formation step wherein the nanoparticles are fluoride-based nanoparticles co-doped with Yb³⁺ and Er³⁺ expressed by the following Chemical Formula 1,

NaY_{1-w-z-x-y}Gd_{w}L_{z}F₄:Yb³⁺ₓ,Er³⁺_{y} (1)

The detailed description of the chemical formula 1 is the same as defined above.

The mixture solution preparation step may comprise a process of preparing a first mixture solution containing a yttrium precursor, a ytterbium precursor, an erbium precursor, oleic acid and 1-octadecene.

The first mixture solution may further comprise a gadolinium precursor.

The yttrium precursor may be any one selected from the group consisting of yttrium acetate (Y(CH₃COO)₃), yttrium chloride (YCl₃), yttrium chloride hexahydrate (YCl₃.6H₂O), and combinations thereof.

The ytterbium precursor may be any one selected from the group consisting of ytterbium acetate (Yb(CH₃COO)₃), ytterbium chloride (YbCl₃), ytterbium chloride hexahydrate (YbCl₃.6H₂O), and combinations thereof.

The erbium precursor may be any one selected from the group consisting of erbium acetate (Er(CH₃COO)₃), erbium chloride (ErCl₃), erbium chloride hexahydrate (ErCl₃.6H₂O), and combinations thereof.

The gadolinium precursor may be any one selected from the group consisting of gadolinium acetate (Gd(CH₃COO)₃), gadolinium chloride (GdCl₃), gadolinium chloride hexahydrate (GdCl₃.6H₂O), and combinations thereof.

The complex formation step may comprise a process of heating the first mixture solution to form a solution containing a lanthanide complex.

The heat treatment may be carried out at temperatures of 100 to 200° C., or carried out at temperatures of 130 to 180° C. In case where the heating is carried out in the range of the above temperatures, a lanthanide oleate complex can be formed.

The reaction solution preparation step may comprise a process of mixing a solution containing the lanthanide complex with a second mixture solution containing a sodium precursor, a fluorine precursor and alcohol to prepare a reaction solution.

The sodium precursor may be any one selected from the group consisting of sodium hydroxide, sodium fluoride, and combinations thereof.

The fluorine precursor may be any one selected from the group consisting of ammonium fluoride, sodium fluoride, and combinations thereof.

In the above, sodium fluoride can serve as both the sodium precursor and the fluorine precursor.

The alcohol may be methanol.

The nanoparticles formation step may comprise a process of removing alcohol from the reaction solution and performing a thermal treatment on the reaction solution from which alcohol is removed to form nanoparticles.

The thermal treatment may be carried out at temperatures of 200 to 370° C. for 30 minutes to four hours, or may be carried out at temperatures of 250 to 330° C. for 60 minutes to two hours under an inert gas atmosphere.

In case where the thermal treatment is carried out within the above temperature and time range, it is possible to form β-phase nano crystalline particles, and exhibit excellent up-conversion emission properties.

In case where the thermal treatment temperature is less than 200° C., it may be possible to obtain particles with an incomplete β-phase single nano crystalline structure, and exhibit insufficient up-conversion emission.

In case where the thermal treatment temperature is greater than 370° C., agglomeration phenomena of particles may occur, and relatively large sized nanoparticles of 20 nm or more may be formed, and the particles size distribution may not be uniform, and the brightness of light-emitting nanoparticles may be reduced.

The nanophosphor preparation method may further comprise a cooling step and a cleaning step subsequent to the nanoparticles formation step.

The cooling step may comprise a process of finishing the thermal treatment process and cooling nanoparticles to room temperature, and the cleaning step may comprise a process of cleaning the cooled nanoparticles with acetone or ethanol.

The nanoparticles may be dispersed and stored in a nonpolar solvent which can be selected from the group consisting of hexane, toluene, chloroform, and combinations thereof, but not limited to them.

The method of preparing a nanophosphor may further comprise a shell formation step subsequent to the nanoparticles formation step.

The shell formation step may comprise a shell solution preparation step, a nanoparticles mixing step, a shell reaction solution preparation step, and a shell formation step.

The shell solution preparation step may comprise a process of preparing a lanthanide precursor containing the gadolinium precursor and a third mixture solution containing sodium oleate.

The gadolinium precursor may be any one selected from the group consisting of gadolinium acetate (Gd(CH₃COO)₃), gadolinium chloride (GdCl₃), gadolinium chloride hexahydrate (GdCl₃.6H₂O), and combinations thereof.

The nanoparticles mixing step may comprise a process of performing a thermal treatment on the third mixture solution to form gadolinium oleate, dissolving the gadolinium oleate in a solution containing oleic acid, 1-octadecene, and mixing nanoparticles that have been formed during the nanoparticles formation step with the solution to prepare a fourth mixture solution.

The nanoparticles that have been formed during the nanoparticles formation step may be nanoparticles formed during the nanoparticles formation step, or nanoparticles that have passed through the cooling or cleaning step.

The shell reaction solution preparation step may comprise a process of mixing a solution containing a sodium precursor, a fluorine precursor and alcohol with the fourth mixture solution to prepare a shell reaction solution.

The sodium precursor may be any one selected from the group consisting of sodium hydroxide, sodium fluoride, and combinations thereof.

The fluorine precursor may be any one selected from the group consisting of ammonium fluoride, sodium fluoride, and combinations thereof.

The alcohol may be methanol.

The shell formation step may comprise a process of removing alcohol from the shell reaction solution and performing a thermal treatment on the shell reaction solution from which alcohol is removed to form a shell on a surface of the core containing the nanoparticles.

The thermal treatment may be carried out at temperatures of 200 to 370° C. for 30 minutes to four hours, or may be carried out at temperatures of 250 to 330° C. for 60 minutes to two hours under an inert gas atmosphere.

In case where the thermal treatment is carried out within the above temperature and time range, β-phase may be formed to epitaxially form a shell, and exhibit excellent up-conversion emission properties even though having a small size of particles when the nanophosphor is formed only with nanoparticles.

In case where the thermal treatment temperature is less than 200° C., it may be difficult to form a shell having β-phase.

In case where the thermal treatment temperature is greater than 370° C., the shell precursor may form a core as well as a shell, and thus the shell may not be effectively formed.

Contrary to the conventional method of preparing fluoride-based nanoparticles co-doped with Yb³⁺ and Er³⁺ having a n-phase structure which is a typical hexagonal structure, a method of preparing a nanophosphor according to the present invention allows the size of particles to be reduced to less than 20 nm, and obtain high luminescent properties excited by infrared rays even though with such a small size of particles, and thus may be suitably applicable to a contrast agent for obtaining in vivo imaging.

Also, it may be difficult to obtain a very small size of nanophosphor without using the process of reducing the temperature or time of synthesis (thermal treatment) to reduce the size of nanophosphor. Further, even if the small sized nanophosphor can be formed by reducing the temperature or time of synthesis (thermal treatment), the crystallinity of the formed nanophosphor may be deteriorated, thereby deteriorating the luminescent properties. However, according to the present invention, it may be possible to exhibit excellent luminescent properties even though having a very small size of particles using a nanophosphor doped with Gd or lanthanide.

A contrast agent according to still another embodiment of the present invention may comprise the nanophosphor. The contrast agent may be a fluorescent contrast agent, or magnetic resonance imaging contrast agent.

The description of the nanophosphor is the same as the foregoing description, and thus the detailed description thereof will be omitted.

A contrast agent containing the nanophosphor may be excited by infrared rays to exhibit a sufficient luminous intensity even through having a small size of particles contained in the contrast agent as much as applicable to in vivo situations even through having a small size of particles, and thus may be used as a fluorescent contrast agent.

Furthermore, a contrast agent containing the nanophosphor may have magnetic properties along with luminescent properties through the property of a nanophosphor contained in the contrast agent, and thus may be used as a magnetic resonance contrast agent.

An infrared sensor according to still yet another embodiment of the present invention may comprise the nanophosphor. The description of the nanophosphor is similar to the foregoing description, and thus the detailed description thereof will be omitted.

The infrared sensor may comprise the nanophosphor being excited by infrared rays to emit light, thereby enhancing a sensitivity of the infrared sensor.

A counterfeit prevention code according to yet still another embodiment of the present invention may comprise the nanophosphor. The description of the nanophosphor is similar to the foregoing description, and thus the detailed description thereof will be omitted.

The counterfeit prevention code may not be easily detected because of using a minute size of nanophosphor invisible to human eyes, and its counterfeit prevention marking can be recognized through the property of being excited by infrared rays to emit light or magnetic properties, and thus may be used as a high level security code.

A solar cell according to still yet another embodiment of the present invention may comprise the nanophosphor. The description of the nanophosphor is similar to the foregoing description, and thus the detailed description thereof will be omitted.

The efficiency of the solar cell may be enhanced by the nanophosphor capable of converting infrared rays into visible light.

The nanophosphor may exhibit an excellent luminous intensity even though having a small size of particles, and be excited by infrared rays to emit visible light, and have magnetic properties and thus can be used as a contrast agent, a counterfeit prevention code, and the like.

### BRIEF DESCRIPTION OF THE DRAWINGS

The accompanying drawings, which are included to provide a further understanding of the invention and are incorporated in and constitute a part of this specification, illustrate embodiments of the invention and together with the description serve to explain the principles of the invention.

In the drawings:

FIG. 1 is the X-ray diffraction pattern of a nanophosphor prepared by Example 1 of the present invention;

FIG. 2 is the transmission electron microscopy and high-resolution transmission electron microscopy photos of a nanophosphor prepared by Example 1 of the present invention;

FIG. 3 is the photoluminescence spectra of a nanophosphor prepared by Example 1 of the present invention;

FIG. 4 is the X-ray diffraction pattern of a nanophosphor made of fluoride-based nanoparticles having a size of 10 nm or less, which are prepared by Example 2 of the present invention;

FIG. 5 is the transmission electron microscopy and high-resolution transmission electron microscopy photos of a nanophosphor made of fluoride-based nanoparticles having a size of 10 nm or less, which are prepared by Example 2 of the present invention;

FIG. 6 is the photoluminescence spectra of a nanophosphor made of fluoride-based nanoparticles having a size of 10 nm or less, which are prepared by Example 2 of the present invention;

FIG. 7 is the schematic diagram illustrating a nanophosphor having a cross-sectional structure made of a core and a shell according to an example of the present invention;

FIG. 8 is the transmission electron microscopy and high-resolution transmission electron microscopy photos of a nanophosphor according to Example 3 of the present invention;

FIG. 9 is a graph illustrating the photoluminescence spectra of a nanophosphor according to Example 3 of the present invention and the photoluminescence spectra of a nanophosphor according to Example 2 of the present invention; and

FIG. 10 is the magnetic hysteresis curve of a nanophosphor according to Example 3 of the present invention.

### BEST MODE FOR CARRYING OUT THE INVENTION

Hereinafter, examples of the present invention will be described in detail with reference to the accompanying drawings to such an extent that the present invention can be easily embodied by a person having ordinary skill in the art to which the present invention pertains. However, the present invention may be implemented in various different forms, and therefore, the present invention is not limited to the illustrated embodiments.

### Example 1

### Preparation of Fluoride-Based Nanoparticles Co-Doped with Yb³⁺ and Er³⁺

0.8 mmol of yttrium chloride hexahydrate (YCl₃.6H₂O), 0.18 mmol of ytterbium chloride hexahydrate (YbCl₃.6H₂O), and 0.02 mmol of erbium chloride hexahydrate (ErCl₃.6H₂O) were mixed with the solvent comprising 6 ml of oleic acid and 15 ml of 1-octadecen to prepare a first mixture solution.

The lanthanide compound was dissolved in the solvent by heating the first mixture solution at 150° C. to form a clear solution, and thus the solution containing a lanthanide complex was formed.

A second mixture solution in which 2.5 mmol of sodium hydroxide and 4 mmol of ammonium fluoride were mixed with methanol was mixed with the said solution containing the lanthanide complex, and then mixed by using a magnetic stirrer to prepare a reaction solution.

Methanol was removed from the reaction solution, and then the reaction solution from which the methanol was removed was thermally treated at 300° C. for 90 minutes under argon atmosphere.

While performing the thermal treatment, a nanophosphor β-NaY_{0.8}F₄:Yb³⁺_{0.18},Er³⁺_{0.02} was formed. The formed nanophosphor was cleaned with ethanol, and dispersed and stored in hexane.

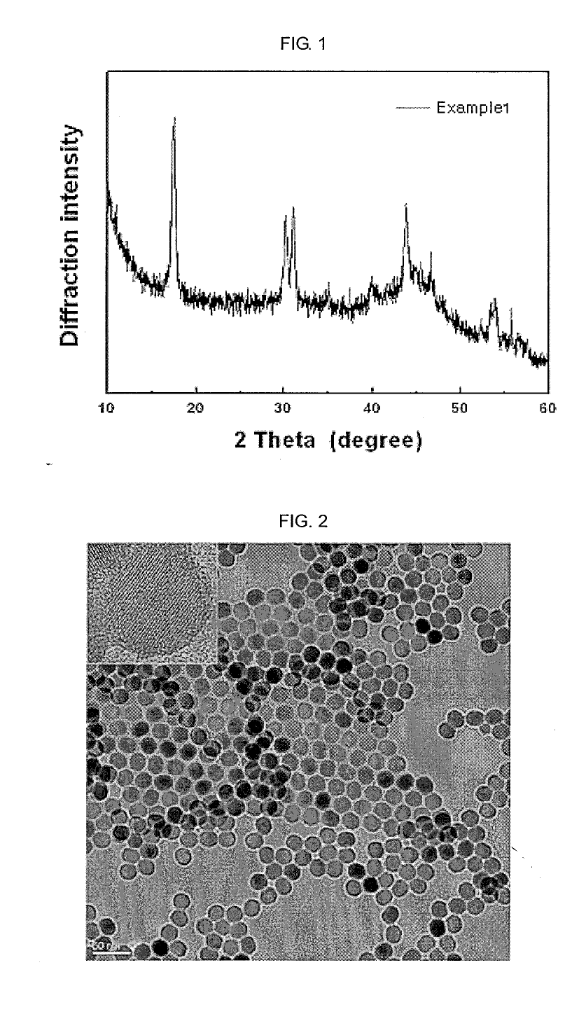

FIG. 1 is the X-ray diffraction pattern of a nanophosphor prepared by Example 1 of the present invention. The X-ray diffraction pattern was measured by using PANalytical's X′pert PRO. Hereinafter, the same equipment was used for the measurement of a X-ray diffraction.

Referring to FIG. 1, it was confirmed that the β-NaY_{0.8}F₄:Yb³⁺_{0.18},Er³⁺_{0.02} nanophosphor was well formed with a hexagonal p-phase NaYF₄ structure.

FIG. 2 is the transmission electron microscopy and high-resolution transmission electron microscopy photos of a nanophosphor prepared by Example 1 of the present invention. The transmission electron microscopy and high-resolution transmission electron microscopy photos were measured by using FEI model TECNAI F20 G². Hereinafter, the same equipment was used for the measurement of the transmission electron microscopy and high-resolution transmission electron microscopy photos.

Referring to FIG. 2, it was confirmed that the size of the nanophosphor β-NaY_{0.8}F₄:Yb³⁺_{0.18},Er³⁺_{0.02} was about 21.5 nm. Furthermore, referring to the high-resolution transmission electron microscopy photo of FIG. 2, it was confirmed that nanophosphor particles synthesized in the Example 1 have a distinctive lattice pattern, and this means that the synthesized nanophosphor has a very high crystallinity.

In general, if the crystallinity of a fluorescent body matrix is high, then the fluorescent body exhibits a high luminescent characteristic, and thus it was found that the nanophosphor β-NaY_{0.8}F₄:Yb³⁺_{0.18},Er³⁺_{0.02} exhibited excellent luminescent properties from such a high crystallinity.

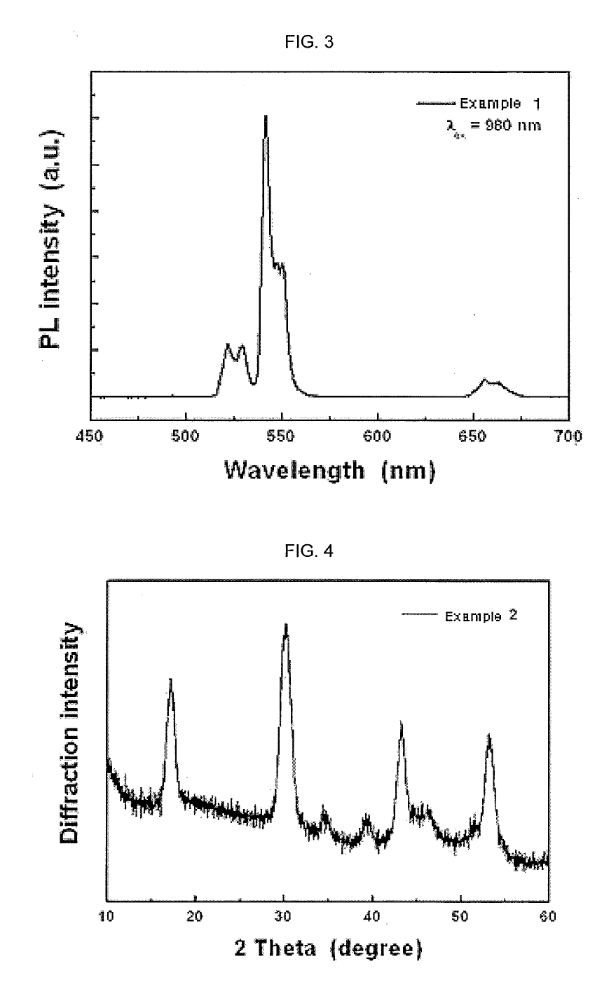

FIG. 3 is the photoluminescence (PL) spectra of a nanophosphor prepared by Example 1 of the present invention. The photoluminescence (PL) was measured by using Hitachi model F-7000. Hereinafter, the same equipment was used for the measurement of photoluminescence (PL).

The photoluminescence spectrum was result of a measurement of the emission spectrum when the nanophosphor synthesized by the Example 1 was excited by near infrared emission at 980 nm, and referring to FIG. 3, it was confirmed that the nanophosphor synthesized by the Example 1 exhibited green emission having a main emission peak centered at 542 nm.

An emission peak around at 542 nm was emitted by electronic transition from the energy level ⁴S_{3/2} of erbium to the energy level ⁴I_{15/2}, and a weak emission peak centered at 525 nm was emitted by electronic transition from the energy level ²H_{11/2} to the energy level ⁴I_{15/2}, and a weak emission peak around 660 nm was emitted by electronic transition from the energy level ⁴F_{9/2} to the energy level ⁴I_{15/2}.

### Example 2

### Preparation of Fluoride-Based Nanoparticles Co-Doped with Yb³⁺ and Er³⁺ and Doped with Gd

0.2 mmol of yttrium chloride hexahydrate (YCl₃.6H₂O), 0.6 mmol of gadolinium chloride hexahydrate (GdCl₃.6H₂O), 0.18 mmol of ytterbium chloride hexahydrate (YbCl₃.6H₂O), and 0.02 mmol of erbium chloride hexahydrate (ErCl₃.6H₂O) were mixed with the solution comprising 6 ml of oleic acid and 15 ml of 1-octadecen to prepare a first mixture solution.

The lanthanide compound was dissolved in the solvent by heating the first mixture solution at 150° C. to form a clear solution, and thus the solution containing a lanthanide complex was formed.

A second mixture solution in which 2.5 mmol of sodium hydroxide and 4 mmol of ammonium fluoride were mixed with methanol was mixed with the solution containing a lanthanide complex, and then mixed by using a magnetic stirrer to prepare a reaction solution.

Methanol was removed from the reaction solution, and then the reaction solution from which the methanol was removed was thermally treated at 300° C. for 90 minutes under argon atmosphere.

While performing the thermal treatment, a nanophosphor β-NaY_{0.8}F₄:Yb³⁺_{0.18},Er³⁺_{0.02} was formed. The formed nanophosphor was cleaned with ethanol, and dispersed and stored in hexane.

FIG. 4 is the X-ray diffraction pattern of a nanophosphor made of fluoride-based nanoparticles having a size of 10 nm or less, which are prepared by Example 2 of the present invention. Referring to FIG. 4, it was confirmed that the nanophosphor β-phase NaY_{0.2}Gd_{0.6}F₄:Yb³⁺_{0.18},Er³⁺_{0.02} prepared by the Example 2 was well formed with a fluoride matrix having a hexagonal phase similarly to the nanophosphor NaY_{0.8}F₄:Yb³⁺_{0.18},Er³⁺_{0.02} prepared by the Example 1.

However, though the nanophosphor synthesized by the Example 2 was synthesized under the same thermal treatment condition as the nanophosphor synthesized by the Example 1, the size of nanophosphor particles was less than that of the nanophosphor synthesized through Example 1, and thus it was seen that the full width at half maximum of a peak exhibited in the X-ray diffraction pattern of FIG. 4 was broad.

In addition, it was confirmed that the diffraction peak position had been moved to a small diffraction angle region since yttrium in the matrix was partially substituted by gadolinium, and as a result of the measurement of FIG. 4, it was confirmed that solid solution NaY_{0.2}Gd_{0.6}F₄ was well formed by the Example 2.

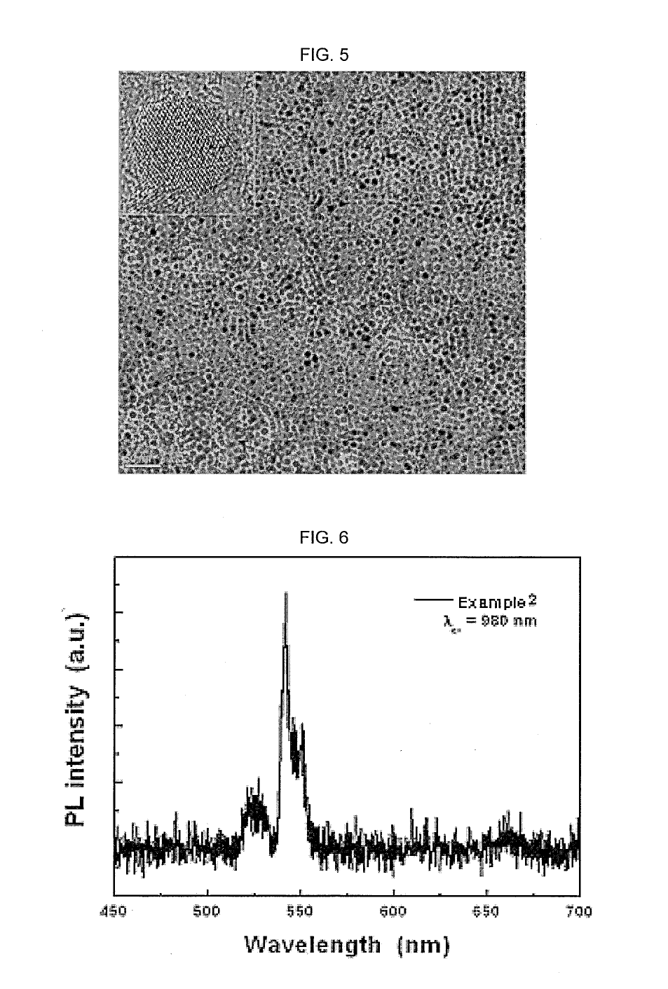

FIG. 5 is the transmission electron microscopy and high-resolution transmission electron microscopy photos of a nanophosphor made of fluoride-based nanoparticles having a size of 10 nm or less, which are prepared by Example 2 of the present invention. Referring to FIG. 5, it was confirmed that the nanophosphor NaY_{0.2}Gd_{0.6}F₄:Yb³⁺_{0.18},Er³⁺_{0.02} prepared by the Example 2 has an average size of 8.3 nm.

Furthermore, the high-resolution transmission electron microscopy photo illustrated in FIG. 5 exhibited a regular crystalline lattice structure, and thus it was confirmed that a nanophosphor having a very high crystallinity had been synthesized.

In other words, considering the preparation method of the Example 2 and the size and crystallinity of a nanophosphor NaY_{0.2}Gd_{0.6}F₄:Yb³⁺_{0.18},Er³⁺_{0.02} prepared by the same method, it was confirmed that according to the present invention, a nanophosphor β-NaY_{0.2}Gd_{0.6}F₄:Yb³⁺_{0.18},Er³⁺_{0.02} exhibiting excellent luminescent properties and having a particle size of 10 nm or less while having a very high crystallinity.

FIG. 6 is the photoluminescence spectra of a nanophosphor made of fluoride-based nanoparticles having a size of 10 nm or less, which are prepared by Example 2 of the present invention. Referring to FIG. 6, it was confirmed that a nanophosphor β-NaY_{0.2}Gd_{0.6}F₄:Yb³⁺_{0.18},Er³⁺_{0.02} prepared by the Example 2 exhibited green emission having an emission peak of 542 nm when the nanophosphor was excited by using a near infrared beam of 980 nm.

### Example 3

### Preparation of Core/Shell Fluoride-Based Nanophosphor Co-Doped with Yb³⁺ and Er³⁺ and Doped with Gd

Using nanoparticles β-NaY_{0.2}Gd_{0.6}F₄:Yb³⁺_{0.18},Er³⁺_{0.02} prepared by the Example 2, a shell was formed on a surface of the core as follows.

0.5 mmol of gadolinium chloride hexahydrate (GdCl₃.6H₂O) and 1.55 mmol of sodium oleate (NaC₁₈H₃₃O₂) were mixed with the mixed solvent of water, ethanol and hexane to prepare a third mixture solution. The third mixture solution was thermally treated at 60° C. for 30 minutes to prepare gadolinium oleate.

The gadolinium oleate was dissolved in a solution containing oleic acid and 1-octadecen, and nanoparticles β-NaY_{0.2}Gd_{0.6}F₄:Yb³⁺_{0.18},Er³⁺_{0.02} prepared by the Example 2 was mixed with the solution containing gadolinium oleate, and then mixed by using a magnetic stirrer to prepare a fourth mixture solution.

5 ml of methanol solution containing 1.25 mmol of sodium hydroxide and 2 mmol of ammonium chloride was mixed with the fourth mixture solution, and then mixed with a magnetic stirrer to prepare a shell reaction solution.

The shell reaction solution was heat-treated similarly to the Example 2 to obtain a nanophosphor having a core/shell structure expressed by β-NaY_{0.2}Gd_{0.6}F₄:Yb³⁺_{0.18},Er³⁺_{0.02}/NaGdF₄ (core/shell). The nanophosphor obtained by the Example 3 was cleaned by using ethanol and then dispersed and stored in hexane.

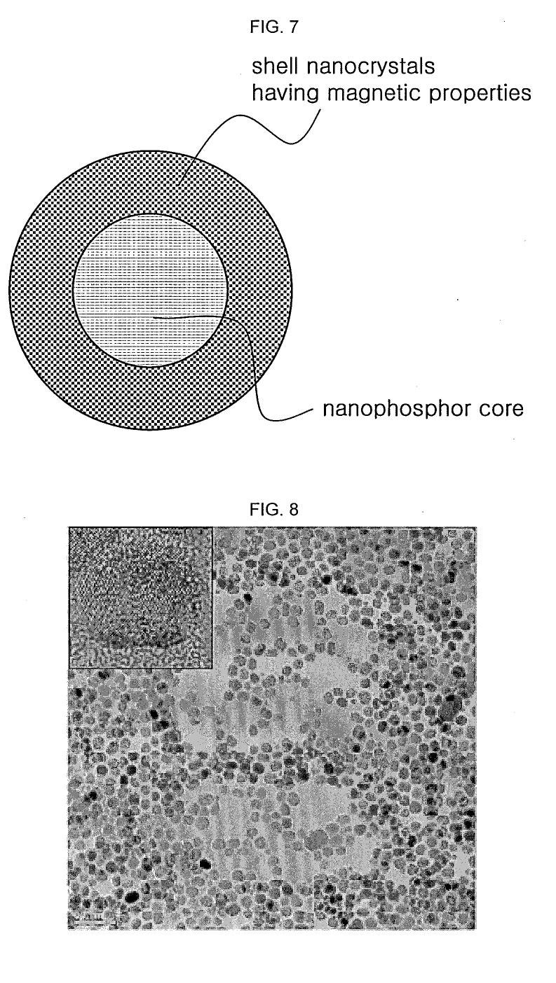

FIG. 7 is the schematic diagram illustrating a nanophosphor having a cross-sectional structure of nanophosphor having a core/shell structure prepared according to the Example 3. Referring to FIG. 7, the nanophosphor may comprise a nanophosphor core and shell nanocrystals having magnetic properties on its surface. The size of nanophosphor core may be 1 to 10 nm, and excited by infrared rays to emit green light. For the shell formed on a surface of the core, the size of the entire nanophosphor may be 15 nm or less even though containing the size of the formed shell, and a nanophosphor having the core/shell structure may exhibit magnetic properties along with luminescent properties.

FIG. 8 is the transmission electron microscopy and high-resolution transmission electron microscopy photos of a nanophosphor according to Example 3 of the present invention. Referring to FIG. 8, it was confirmed that a nanophosphor β-NaY_{0.2}Gd_{0.6}F₄:Yb³⁺_{0.18},Er³⁺_{0.02}/NaGdF₄ having a core/shell structure prepared by the Example 3 contained nanoparticles prepared by the Example 2 as its core, and a shell was formed around the core to increase the size of nanophosphor, and its average size was 12.8 nm.

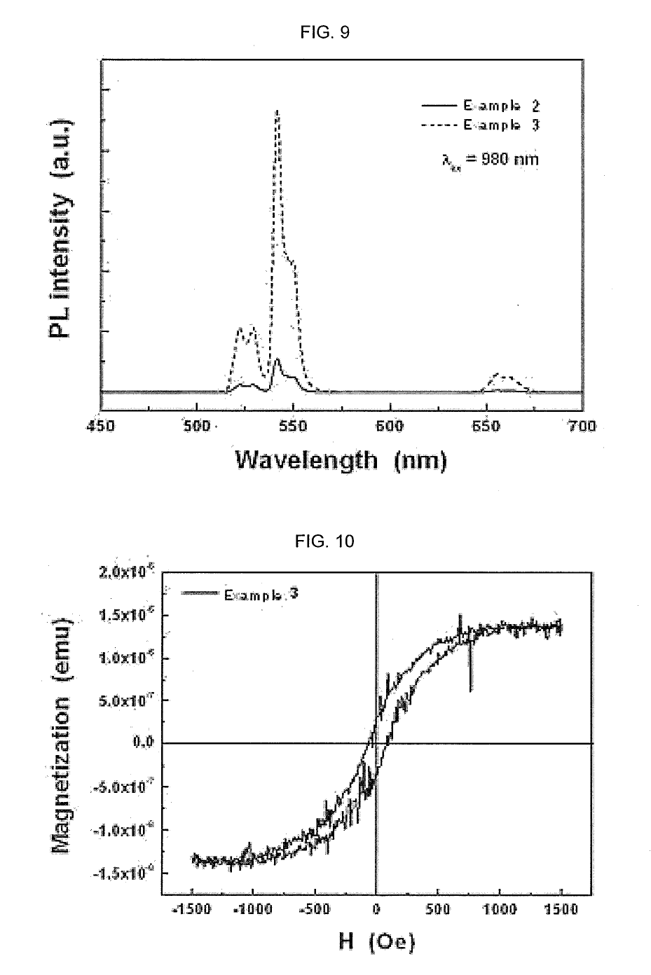

FIG. 9 is a graph illustrating the photoluminescence spectra of a nanophosphor according to Example 3 of the present invention and the photoluminescence spectra of a nanophosphor according to Example 2 of the present invention.

Referring to FIG. 9, a nanophosphor β-NaY_{0.2}Gd_{0.6}F₄:Yb³⁺_{0.18},Er³⁺_{0.02}/NaGdF₄ having a core/shell structure prepared by the Example 3 exhibits a luminous intensity of eight times stronger than that of the nanophosphor β-NaY_{0.2}Gd_{0.6}F₄:Yb³⁺_{0.18},Er³⁺_{0.02} prepared by the Example 2.

FIG. 10 is the magnetic hysteresis curve of a nanophosphor according to Example 3 of the present invention. The magnetic hysteresis curve was measured by using Princeton model Mag 2900.

Referring to FIG. 10, it was confirmed that the nanophosphor β-NaY_{0.2}Gd_{0.6}F₄:Yb³⁺_{0.18},Er³⁺_{0.02}/NaGdF₄ having a core/shell structure exhibited super-paramagnetic properties.

Referring to FIGS. 9 and 10, it was confirmed that a nanophosphor having a core/shell structure according to an example of the present invention exhibited intense green emission under near infrared excitation as well as magnetic properties.

In addition, by comparing FIG. 2 and FIG. 5, it was confirmed that the size of nanoparticles can be drastically reduced in case of the Example 2 (FIG. 5) compared to the Example 1 (FIG. 2) of the present invention. Moreover, in case of a core/shell structure of the Example 3, it shows an excellent effect that the size of particles is drastically smaller while exhibiting luminous intensity of eight times greater than that of the Example 1 and exhibiting magnetic properties that are not shown in the Example 1, and as a result, it may be possible to provide a nanophosphor that can be highly used as a contrast agent as well as applicable to various uses in many areas.

Although the preferred examples of the present invention have been described in detail, the rights scope of the present invention is not limited to the examples and various modifications and improvements thereto made by those skilled in the art using the basic concept of the present invention as defined in the accompanying claims will fall in the rights scope of the invention.

What is claimed is:

1. A nanophosphor comprising fluoride-based nanoparticles co-doped with Yb³⁺ and Er³⁺ expressed by the following Chemical Formula 1,
NaY_{1-w-z-x-y}Gd_{w}L_{z}F₄:Yb³⁺ₓ,Er³⁺_{y} (1)
Wherein,
x is a real number in the range of 0.1≦x≦0.9; y is a real number in the range of 0<y≦0.1; and 0.1<x+y≦1;
w is a real number in the range of 0≦w≦1; and z is a real number in the range of 0≦z≦1; and 0≦w+z≦1; and
L is any one selected from the group consisting of lanthanide elements, and combinations thereof.

2. The nanophosphor of claim 1, wherein the nanophosphor comprises a core containing said nanoparticles and a shell located on a surface of the core, and
the shell comprises the following Chemical Formula 2,
NaGd₁₋ᵥMᵥF₄ (2)
wherein,
v is a real number in the range of 0≦v<1; and
M is any one selected from the group consisting of Y, lanthanide elements, and combinations thereof.

3. The nanophosphor of claim 1 or 2, wherein the lanthanide element is any one selected from the group consisting of La, Ce, Pr, Nd, Pm, Sm, Eu, Gd, Tb, Dy, Ho, Tm and Lu.

4. The nanophosphor of claim 1, wherein said x is a real number in the range of 0.1≦x≦0.4, and said y is a real number in the range of 0.001≦y≦0.05, and 0.101≦x+y≦0.45.

5. The nanophosphor of claim 1, wherein the size of nanoparticles is 1 nm to 10 nm.

6. The nanophosphor of claim 1, wherein the nanoparticles has a hexagonal structure.

7. The nanophosphor of claim 2, wherein the size of nanophosphor is greater than 1 nm and equal to or less than 20 nm.

8. The nanophosphor of claim 2, wherein the nanophosphor has up-conversion properties and magnetic properties.

9. A method of preparing a nanophosphor, the method comprising:
a mixture solution preparation step for preparing a first mixture solution containing a yttrium precursor, a ytterbium precursor, an erbium precursor, oleic acid and 1-octadecene;
a complex formation step for heating the first mixture solution to form a solution containing a lanthanide complex;
a reaction solution preparation step for mixing a solution containing the lanthanide complex with a second mixture solution containing a sodium precursor, a fluorine precursor and alcohol to prepare a reaction solution; and
a nanoparticles formation step for removing alcohol from the reaction solution and performing a thermal treatment on the reaction solution from which alcohol is removed to form nanoparticles,
wherein the nanoparticles are fluoride-based nanoparticles co-doped with Yb³⁺ and Er³⁺ expressed by the following Chemical Formula 1,
NaY_{1-w-z-x-y}Gd_{w}L_{z}F₄:Yb³⁺ₓ,Er³⁺_{y} (1)
Wherein,
x is a real number in the range of 0.1≦x≦0.9; y is a real number in the range of 0<y≦0.1; and 0.1<x+y≦1;
w is a real number in the range of 0≦w≦1; and z is a real number in the range of 0≦z≦1; and 0≦w+z≦1; and
L is any one selected from the group consisting of lanthanide elements, and combinations thereof.

10. The method of claim 9, wherein the first mixture solution further comprises a gadolinium precursor.

11. The method of claim 9, wherein the yttrium precursor is any one selected from the group consisting of yttrium acetate (Y(CH₃COO)₃), yttrium chloride (YCl₃), yttrium chloride hexahydrate (YCl₃.6H₂O), and combinations thereof, and
the ytterbium precursor is any one selected from the group consisting of ytterbium acetate (Yb(CH₃COO)₃), ytterbium chloride (YbCl₃), ytterbium chloride hexahydrate (YbCl₃.6H₂O), and combinations thereof, and
the erbium precursor is any one selected from the group consisting of erbium acetate (Er(CH₃COO)₃), erbium chloride (ErCl₃), erbium chloride hexahydrate (ErCl₃.6H₂O), and combinations thereof.

12. The method of claim 10, wherein the gadolinium precursor is any one selected from the group consisting of gadolinium acetate (Gd(CH₃COO)₃), gadolinium chloride (GdCl₃), gadolinium chloride hexahydrate (GdCl₃.6H₂O), and combinations thereof.

13. The method of claim 9, wherein the thermal treatment performed during the nanoparticles formation step is carried out at temperatures of 200 to 370° C. for 30 minutes to four hours.

14. The method of claim 9, wherein the nanophosphor preparation method further comprises:
a cooling step for cooling the thermally treated nanoparticles, and a cleaning step for cleaning the cooled nanoparticles with acetone or ethanol, subsequent to the nanoparticles formation step.

15. The method of claim 9, further comprising:
a shell formation step subsequent to the nanoparticles formation step, and
the shell formation step comprises:
a shell solution preparation step for preparing a third mixture solution comprising sodium oleate and lanthanide precursor including gadolinium precursor;
a nanoparticles mixing step for performing a thermal treatment on the third mixture solution to form gadolinium oleate, dissolving the gadolinium oleate in a solution containing oleic acid and 1-octadecene, and mixing nanoparticles that have been formed during the nanoparticles formation step with the solution to prepare a fourth mixture solution;
a shell reaction solution preparation step for mixing a solution containing a sodium precursor, a fluorine precursor and alcohol with the fourth mixture solution to prepare a shell reaction solution; and
a shell formation step for removing alcohol from the shell reaction solution and performing a thermal treatment on the shell reaction solution from which alcohol is removed to form a shell on a surface of the core containing the nanoparticles.

16. The method of claim 15, wherein the gadolinium precursor is any one selected from the group consisting of gadolinium acetate (Gd(CH₃COO)₃), gadolinium chloride (GdCl₃), gadolinium chloride hexahydrate (GdCl₃.6H₂O), and combinations thereof.

17. A fluorescent or magnetic resonance imaging contrast agent comprising the nanophosphor of claim 1.

18. An infrared sensor comprising the nanophosphor of claim 1.

19. A counterfeit prevention code comprising the nanophosphor of claim 1.

20. A solar cell comprising the nanophosphor of claim 1.
